(19) United States
(12) Patent Application Publication
(10) Pub. No.: US 2022/0411537 A1
(43) Pub. Date: Dec. 29, 2022

(54) BIO-NANOCOMPOUND AS AN AGENT FOR NUCLEATING AQUEOUS-BASED COMPOUNDS AND PRODUCTION METHOD THEREOF

(71) Applicant: UNIVERSIDAD DE LOS ANDES, Bogota (CO)

(72) Inventors: Isabel PULIDO ESPINOSA, Bogota (CO); Diana Paola CAMACHO SARMIENTO, Bogota (CO); Laura Camila CALDERON ARIAS, Bogota (CO); Juan Carlos ANGULO LOPEZ, Bogota (CO); Johann Faccelo OSMA CRUZ, Bogota (CO); Giovanna DANIES TURANO, Bogota (CO); Carolina OBREGON TARAZONA, Bogota (CO)

(21) Appl. No.: 17/781,255

(22) PCT Filed: May 5, 2021
(86) PCT No.: PCT/IB2021/053817

§ 371 Date: May 31, 2022

Related U.S. Application Data
(60) Provisional application No. 63/020,616, filed on May 6, 2020.

(51) Int. Cl. C07K 17/14; C07K 1/04; B82Y 5/00

(52) U.S. Cl. CPC C07K 17/14; C07K 1/04; B82Y 5/00

(57) ABSTRACT

A bio-nanocompound incorporating metal oxide particles as substrate, an amino organosilane as linker, a dialdehyde as crosslinking agent and a nucleation agent that utilizes ice nucleating activity to create ice on low energy demand at temperatures down to 0° C. and extend cold chains without increasing energy consumption, wherein even the first application is capable of freezing to different shapes and volumes; a method for producing the bio-nanocompound by self-assembly technique, by mixing the substrate, immobilizing the linker on the substrate, immobilizing the crosslinker on the linker and immobilizing by covalent bonding nucleating agent on the crosslinker; and a coolant having the bio-nanocompound.

### TECHNICAL FIELD

This disclosure is in the field of nano-engineering and in the freezing industry, thermal preservation and refrigeration technologies in general. Specifically, the development is directed to a bio-nanocompound for nucleating aqueous-based compounds and the production method thereof. More specifically, the disclosure relates to a new bio-nanocompound having ice nucleation capability to create ice with low energy demand, as well as a first application, which is capable of freezing to different shapes and volumes. The development also relates to a new method for creating ice at temperatures down to 0° C.

### DESCRIPTION OF THE PRIOR ART

The refrigeration or freezing industry requires high energy consumption to maintain the cold chains of perishable products. This, together with the high operating cost of maintaining refrigeration, the limited availability of such solutions in areas of difficult access, the negative environmental impact and the limitation generated by access to electricity, has driven the development of technologies aimed at ice production where electricity is not used and the associated operating costs can be reduced.

In this regard, technologies have been described making it possible to raise the freezing point of water. Although it is known that water freezes at 0° C.; in reality, water without impurities (dust, pollen or bacteria) that function as ice nuclei begins to freeze at a temperature of −41° C. (−41.8° F.). However, water freezing is catalyzed between −2° C. and −4° C. in presence of an ice nucleation protein (INP) [Kang et al. (1999). Ice Nucleation Active Microorganisms. (U.S. Pat. No. 5,972,686A). or INA (Ice Nucleation Agent) proteins] that allows water to nucleate forming ice crystals at temperatures higher than those at which this procedure normally occurs.

According to Mohler, O. et al [Mohler, O., et al. (2008). Heterogeneous ice nucleation activity of bacteria: New laboratory experiments at simulated cloud conditions. Biogeosciences, 5(5), 1425-1435. doi:10.5194/bg-5-1425-2008], one of the most active heterogeneous ice nuclei are INPs, which are commonly found in the cell membrane of the bacterial species Pseudomonas syringae, Pseudomonas biridiflava and Erwinia herbicola. These microorganisms naturally create snow, hail and rain, and are considered plant pathogens, as they can break down plant tissue using their ice nucleation proteins. Pseudomonas syringae contains the InaZ protein, which is the most efficient ice nucleating agent widely used in industry.

The INA protein is a membrane protein comprising approximately 1,200 amino acids and comprises three domains, an N-terminal domain of up to 19 KDa, a large central repeat domain (CRD) of up to 94 KDa and a C-terminal domain of up to 7 KDa [Kawahara, H. (2008). Cryoprotectants and ice-binding proteins. In R. Margesin, F. Schinner, J.-C. Marx, & C. Gerday (Eds.), Psychrophiles: From biodiversity to biotechnology (pp. 229-246). Berlin Heidelberg: Springer. Retrieved from http://link.springer.com/chapter/10.1007/978-3-540-74335-4_14), where CRD is believed to be responsible for ice nucleation ability [Schmid, D., Pridmore, D., Capitani, G., Battistutta, R., Neeser. J.-R., & Jann, A. (1997). Molecular organization of the ice nucleation protein InaV from Pseudomonas syringae. FEBS Letters, 414(3), 590-594. https://doi.org/10.1016/S0014-5793(97)01079-X]. Specifically, the core domain is composed of 50 to 80 tandem repeats of 16 amino acids and each repeat is composed of the consensus amino acid sequence GYGSTxTAxxxSxLxA where x can be any amino acid [Ling, M. et al. (2018). Effects of ice nucleation protein repeat number and oligomerization level on ice nucleation activity. Journal of Geophysical Research: Atmospheres, 123, 1802-1810. https://doi.org/10.1002/2017JD027307].

According to the modeling study of Graether and Jia [Steffen P. Graether, Zongchao Jia. (2001). Modeling Pseudomonas syringae Ice-Nucleation Protein as 43-Helical Protein, Biophysical Journal, Volume 80, Issue, Pages 1169-1173, ISSN 0006-3495, https://doi.org/10.1016/S0006-3495(01)76093-6], the CDR domain has a (β-helical fold and interacts with water through the repetitive motif TxT, which “essentially can pair with the ice network and participate in the hydrogen bonding network by replacing the corresponding oxygen atom of the ice section,” similar to antifreeze proteins (AFP), but with a larger ice-interaction surface area.

INA proteins from P. syringae are used for different applications. However, the most common is snow production at higher temperatures. SNOMAX® is a US company that has extracted the INA protein and industrialized its production for use in ice rinks (optimizing energy consumption). U.S. Pat. No. 6,151,902 describes an invention related to a method of industrializing the production of INA proteins, in order to create a product that functions as a snow inducer, providing additional nuclei to improve the crystallization process and a core of each water droplet, thus transforming it into snow and reducing evaporation. This publication mentions that P. syringae proteins can freeze water at an average temperature of −2° C., with the highest freezing temperature being −0.6° C./31° F. However, they do not guarantee that freezing will always occur at the same temperature.

In addition, immobilization of ice nucleating agents has been developed to increase freezing efficiency at higher temperatures and to ensure longer lasting ice. International patent application WO 2018/005802, discloses ice nucleation formulations for the preservation of biological products, wherein the ice nucleation agent (INA particles) is encapsulated or embedded in beads of a hydrogel. Said hydrogel is used to cryopreserve and stabilize cells, cell tissues, lipids, nucleic acids, in some exosomes and organs and freeze different liquids such as water, glycerol or heavy water at temperatures ranging from 5° C. to 1° C.

In particular, this technology uses alginate and agarose microencapsulation and nanoencapsulation of ice nucleating agents such as INA proteins or mineral nucleating agents such as IceStart®, where microencapsulation allows the INA proteins to remain in their respective capsules while dispersed in the organs or cells and, once their function as ice nucleators is fulfilled, they can be removed from the surface of each biological sample.

However, reuse of the ice nucleation agent is not disclosed, making this an expensive technology that is discarded once it has been used, due to the protein natural degradation. The fact that this method of immobilization by microencapsulation does not maintain the INA protein structure, explains why each ice nucleation agent will degrade and its nucleating activity will be lost at different temperatures.

On the other hand, patent EP 1829890 discloses a product having ice nucleation capability and a method for producing ice with low energy by immobilizing polypeptides such as antifreeze proteins, peptides and oligopeptides on a vehicle that may include conductive materials, such as bead-like metals (nanoparticles or macroparticles), or planar surfaces, among others. The method includes, generally, binding between residues introduced into the polypeptides and vehicles, such as binding the polypeptide on the vehicle surface with a silane coupling agent having an epoxy group. This method is able to raise the freezing point of water to −2° C. by immobilizing INA proteins. However, the ice nucleating active vehicle has a certain number of times until it loses ice nucleation ability and does not provide stronger ice, because the ice nucleators are not homogeneously distributed in water. Therefore, only the water in contact with the vehicle will freeze at −2° C., while the remaining water, if frozen, will melt at a faster rate.

Only one of the aforementioned freezing methods creates ice above 0° C., which means that energy consumption remains high. Furthermore, although these methods are contributing to the creation of more resistant ice, they are not making a significant difference in reducing the energy consumption for ice formation, due to the use of INA proteins. On the other hand, the contexts where INA proteins are used are mainly in industrial snow production with machines introducing extreme pressure changes to make ice crystals below 0° C. Additionally, none of these inventions provides a more robust and reusable ice nucleator with the ability to freeze water at 1.5-2.5° C.

Therefore, the high energy consumption to preserve cold chains requires the development of alternatives that not only reduce the energy required to reach the freezing point, but also extend the cold chain cycle without increasing energy consumption. In addition, there is still a need to implement technologies to recover and reuse INA proteins, increase their stability when placed on substrates and in the event of temperature changes above 10° C., and control the amount of free INA proteins to guarantee a stable freezing temperature.

This disclosure provides a more efficient, resilient and durable ice nucleator at temperatures down to 0° C. that may result in decreased energy consumption during ice formation. The bio-nanocompound with INA proteins of the disclosure retains high activity during the first three uses. After the third use, it decreases exponentially. However, the usability of this disclosure could provide a more stable cooling chain, because it provides a higher melting point and can be reused for a number of times, optimizing INA protein consumption.

### BRIEF DESCRIPTION

This disclosure refers to a bio-nanocompound comprising a metal oxide particulate substrate, a linker, a crosslinking agent and an ice nucleation agent.

Additionally, a self-assembly method is developed for the preparation of said bio-nanocompound comprising the steps of mixing the metal oxide substrate, immobilizing the linker, immobilizing the crosslinking agent and immobilizing by covalent bonding the ice nucleation agent, in order to obtain a product intended for nucleating aqueous-based compounds at temperatures higher than the freezing temperature of the medium and increasing the thawing time of the medium.

### BRIEF DESCRIPTION OF THE FIGURES

FIG. 1 Bio-nanocompound assembly. General configuration of the bio-nanocompound structure, wherein the bio-nanocompound comprises a substrate of metal oxide particles with a size in the nanometer to submillimeter range (1); a linker (2); a crosslinker (3); and an ice nucleation agent (4).

FIG. 2 Molecular structure of the bio-nanocompound. The APTES molecule functions as the linker and glutaraldehyde as the crosslinker between the nanoparticles and the INA protein. A. Aluminum oxide bio-nanocompound; B. Magnetite bio-nanocompound; C. Silicon dioxide bio-nanocompound.

FIG. 3 Mean nanoparticle size obtained from the ratio of percent intensity to size in nanometers, with three replicas. A. magnetite=132 nm. B. silicon dioxide=2243 nm.

FIG. 4 Immobilization efficiency. Silicon dioxide nanoparticles have the highest immobilization efficiency and therefore those with the highest amount of INA protein on their surface. The efficiency values are 99.99%, 99.87% and 99.80% for silicon dioxide, aluminum oxide and magnetite, respectively. These values were obtained with the following formula: immobilization efficiency=[(100)−(supernatant concentration x supernatant volume×100)/initial amount of protein)].

FIG. 5 Silicon dioxide thawing activity. Example 6 (Experiment 1). Thawing temperature variation was measured for the 3 replicas of the bio-nanocompound at a concentration of 2 mg/ml for one minute at room temperature (23° C.).

FIG. 6 State analysis. Example 6 (Experiment 2). A. Magnetite; B. Silicon dioxide; C. Free INA protein. The state of different concentrations of silicon dioxide and magnetite bio-nanocompounds was analyzed over time in seconds. Comparison of type I water control and free INA protein is included.

FIG. 7. Temperature analysis. Example 6 (Experiment 2). The temperature variation of different concentrations of silicon dioxide and magnetite bio-nanocompounds was analyzed over time in seconds. Comparison of type I water control and free INA protein is included. This figure includes FIGS. 7A, 7B and 7C, which correspond individually to magnetite, silicon dioxide and free INA, respectively.

FIG. 8. State analysis. Example 6 (Experiment 2). The state of different concentrations of silicon dioxide and magnetite bio-nanocompounds was analyzed a second time over time in seconds. Control comparison of type I water and free proteins is included. Each sample had 3 replicas and the values presented in the graph correspond to the average of the values obtained. This figure includes FIGS. 8A, 8B and 8C, which correspond individually to magnetite, silicon dioxide and free INA, respectively.

FIG. 9. Temperature analysis. Example 6 (Experiment 2). The temperature variation of different concentrations of silicon dioxide and magnetite bio-nanocompounds was analyzed over time in seconds for the second time. Control comparison of type I water and free INA protein is included, and each sample had 3 replicas. Each sample had 3 replicas and the values presented in the graph correspond to the average of the values obtained. This figure includes FIGS. 9A, 9B and 9C, which correspond individually to magnetite, silicon dioxide and free INA, respectively.

FIG. 10. Minimum freezing temperature. Example 7. Each sample was subjected to different temperatures inside a freeze dryer to establish the minimum freezing temperature of the bio-nanocompounds, the free INA protein and the water control.

FIG. 11. Thawing activity. Example 6 (Experiment 3). The thawing time was measured for those samples that showed freezing activity at −1.1° C. inside a freeze dryer. Samples A through C correspond to magnetite, D through F correspond to silicon dioxide, and G through I correspond to free INA protein. Figure A includes one replica of 1 mg/ml and two of 0.5 mg/ml, B two of 2 mg/ml and three for the rest of the treatments and C two replicas of 0.1 mg/ml and 0.05 mg/ml. This figure includes FIGS. 11A, 11B and 11C, which correspond individually to magnetite, silicon dioxide and free INA protein, respectively.

FIG. 12. Thawing activity. Example 6 (Experiment 4). Thawing time in minutes was measured for samples that were frozen at −5° C. (freeze dryer temperature) and −2.8° C. (internal temperature). This figure includes FIGS. 12A, 12B, and 12C, which correspond individually to magnetite, silicon dioxide, and free INA protein, respectively.

FIG. 13. Thawing activity. Example 6 (Experiment 5). Thawing time in minutes was measured for all samples that were frozen at −6° C. (freeze dryer temperature) or −4.1° C. (internal temperature). This figure includes FIGS. 13A, 13B, and 13C, which correspond individually to magnetite, silicon dioxide, and free INA protein, respectively.

FIG. 14. Thawing activity. Example 8. Thawing time in minutes was measured for cycles one and two. This figure includes FIGS. 14A, through 14F, which correspond individually to magnetite cycles one and two, silicon dioxide cycles one and two and free INA protein cycles one and two, respectively.

FIG. 15. Thawing activity. Example 8. Thawing time in minutes was measured for cycles three and four. This figure includes FIGS. 15A to 15F, which correspond individually to magnetite cycles three and four, silicon dioxide cycles three and four, and free INA protein cycles three and four, respectively.

### DETAILED DESCRIPTION

For purposes of interpreting terms used throughout this document, their usual meaning in the technical field should be considered, unless a particular definition is incorporated or the context clearly indicates otherwise. In addition, terms used in the singular form shall also include the plural form.

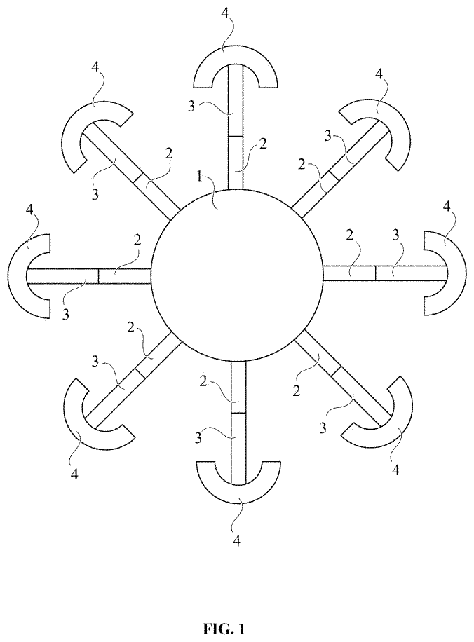

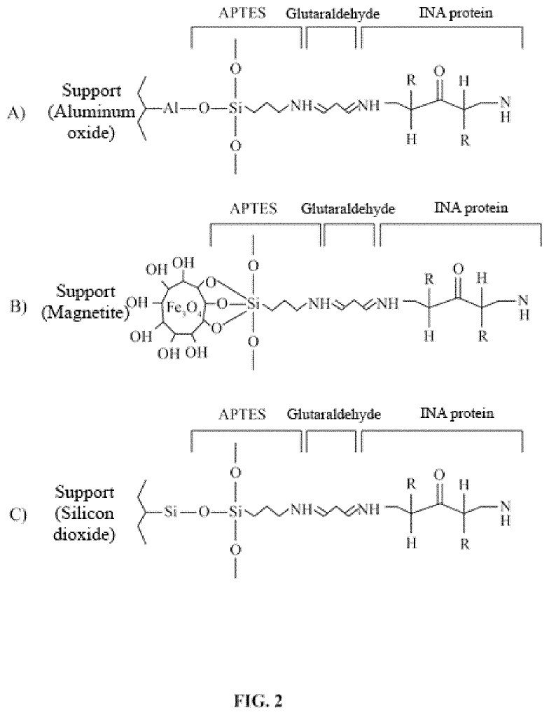

In a first aspect, this disclosure relates to a bio-nanocompound comprising a substrate of metal oxide particles with a size in the nanometer to submillimeter range (1); a linker of an amino organosilane (2); a dialdehyde crosslinking agent or crosslinker (3); and an ice nucleation agent or INA protein (4); wherein the linker is directly attached to the substrate, the crosslinking agent is directly attached to the linker, and the ice nucleation agent is directly attached to the crosslinker. The structure of the bio-nanocompound is schematically illustrated in FIG. 1. The bio-nanocompound allows nucleating crystals of aqueous-based compounds at higher temperatures than the freezing temperature or increase the thawing time of the medium.

Nucleation of water-based compounds is the slow stage of crystallization, when molecules of the liquid begin to spontaneously arrange themselves into a crystal lattice and begin to recruit other molecules to join and agglomerate.

### Bio-Nanocompound

For purposes of this disclosure, a bio-nanocompound is understood to be a nanocompound having at least one biomolecule within its structure. In this disclosure the bio-nanocompound comprises a substrate, a linker, a crosslinker and an ice nucleation agent or protein INA.

Substrate for the purposes of this disclosure is understood as a surface or support material characterized by being of a metal oxide type chemical nature, which is in the form of a flat, spherical, ovoid or irregular surface. In the last case it is referred to as a particle, which has at least one dimension between the nanometer and macrometer scale.

In a particular embodiment, the substrate is selected from, but not limited to, particles of an iron oxide, aluminum, silicon or mixtures thereof. In another particular embodiment, the particles have a size in the nanometer to submillimeter range (1 nm to 1 mm) in at least one of their dimensions. In another particular embodiment, the substrate is particles with diameters between 1 nm and 1 mm, preferably between 100 nm and 500 μm.

The linker is understood as a compound that allows to bind the molecules of the bio-nanocompound to the substrate, through a strong anchor. In one embodiment said linker is an amino organosilane. In another particular embodiment, it is selected from, without limitation, 3-(aminopropyl)triethoxysilane (APTES) or (aminopropyl)trimethoxysilane (APTMS).

The concentration of the linker is determined according to the surface area of the substrate. The calculation is made according to the ratio between the area of the linker and the surface area of the substrate. Preferably, the linker is found in the bio-nanocompound at a concentration on the substrate surface of between 10⁻²⁴ moles/nm² and 10⁻²⁰ moles/nm².

The crosslinking agent or crosslinker for purposes of this disclosure is understood to be a compound that allows binding the linker to the ice nucleation agent. In one embodiment the crosslinking agent is a dialdehyde. In another embodiment, it is selected from, without limitation, glutaraldehyde, glyoxal and succinaldehyde.

The concentration of the crosslinking agent is determined according to the concentration of the linking agent used, so that the molar concentration of the crosslinker is equal to that of the linker.

For purposes of this disclosure, ice nucleation agent means a biomolecule that induces the formation and growth of ice crystals at higher temperatures, when added to an aqueous system. In one embodiment the ice nucleation agent is an INA protein derived from bacteria, fungi, insects or crustaceans.

In a particular embodiment the INA protein is derived from bacteria of the genera Pseudomonas, Erwinia or Xanthomonas, preferably from the cell membrane of Pseudomonas syringae, Pseudomonas viridiflava or Erwinia herbicola.

In another particular embodiment, the INA protein is Z-type from Pseudomonas syringae, W-type from Pseudomonas fluorescens, E-type from Erwinia herbicola, U-type from Erwinia ananas and X-type from Xanthomonas campestris. In one particular embodiment the INA protein is derived from Pseudomonas syringae.

In a preferred embodiment, the protein is a membrane protein comprising approximately 1200 amino acids and is composed of three domains, an N-terminal domain of up to 19 KDa, a large central repeat domain (CRD) of up to 94 KDa and a C-terminal domain of up to 7 KDa (Kawahara, 2008), wherein the CRD is believed to be responsible for ice nucleation activity (Schmid et al., 1997). Specifically, the core domain is composed of 50 to 80 tandem repeats of 16 amino acids and each repeat is composed of the consensus amino acid sequence GYGSTxTAxxxSxLxA where x can be any amino acid (Ling et al, 2018). In a preferred embodiment the protein is a Z-type INA protein.

The concentration of the INA protein is determined according to the concentration of the linker agent used, so that the molar concentration of the INA protein is equal to that of the linker.

The inventors have found that it is possible to immobilize the INA protein on nanoscale metal oxide surfaces: aluminum oxide, magnetite (iron oxide) and silicon dioxide by means of a self-assembly technique. The bio-nanocompound obtained by this technique allows the induction of water freezing when the bio-nanocompound comes in contact with an aqueous medium, due to the maintenance and enhancement of the CDR domain of the proteins.

Water freezing is generated by the “ice nucleation ability” of the INA protein, which means the activity of the protein CDR domain, interacting with water through the TxT motif and participating in the hydrogen bonding network. Freezing activity refers to the promotion of ice nuclei at higher temperatures from the natural freezing point of water.

### Preparation Method of a Bio-Manocompound

In a second aspect, this development is directed to the preparation method of a bio-nanocompound for nucleating aqueous-based compounds comprising the steps of mixing a metal oxide substrate with a solvent medium, immobilizing an amino organosilane linker onto the substrate, immobilizing a dialdehyde crosslinking agent on the linker, and immobilizing by covalent bonding an ice nucleation agent or INA protein on the crosslinker, by means of a chemical anchor that allows to bind the protein to a specific part of its structure without altering its functionality. Complementarily and optionally, it is also possible to wash the resulting bio-nanocompound. The method is carried out by means of a self-assembly or molecular scaffolding technique that immobilizes each of the elements that are part of it, to provide a structure that has a specific orientation allowing it to fulfill its function.

It is essential that the surface chemistry used (self-assembly process) is done in a specific order given the particular chemical structural features of each element that is part of the bio-nanocompound, where the point concentrations allow the construction that results from the self-assembly process to occur spontaneously and remain organized, in terms of ensuring that the structure of the bio-nanocompound at the end of the process is with high probability each time it is obtained.

The immobilization of the elements is carried out due to the specific chemical nature of each element, as described in the bio-nanocompound. In a particular embodiment, the linker is attached to the substrate by a covalent bond and at its extreme part opposite to the substrate bond it contains a terminal amino group, which is directly attached to the crosslinking agent by one of its aldehyde-type terminals, which spontaneously reacts with the amino group through a strong lock-key type bond without involving a great energetic effort. At the opposite end the crosslinking agent has another aldehyde group, which binds to the amino terminus of the INA protein through the same lock-key mechanism described for the crosslinker bond.

This particular configuration allows the INA protein to be chemically immobilized on the surface of the metal oxides through a covalent bond that confers a strong anchorage on the substrate, making it difficult to detach and thus providing greater stability.

Substrate preparation is carried out by mixing the metal oxide particulate substrate with an aqueous solvent medium, preferably deionized water.

Optionally, a dispersant may be added to the substrate solution when the substrate has a particle size in the nanometer range, which is selected from an ionically charged compound, preferably it is tetramethylammonium hydroxide (TMAH). The dispersant is prepared by dissolving it in an aqueous solvent medium which is preferably ultrapure water.

Subsequently the linker is prepared by dissolving the amino organosilane in an aqueous solvent, preferably deionized water, then mixed with the previously prepared substrate solution and said mixture is stirred for a time of at least 10 minutes and a temperature of at least 30° C. The resulting solution is then allowed to stand for at least 10 minutes at least at 15° C. The linker concentration is determined according to the surface area of the substrate. The calculation is made according to the ratio between the area of the linker and the surface area of the substrate. Preferably, the linker is found in the bio-nanocompound at a concentration on the substrate surface of between 10⁻²⁴ moles/nm² and 10⁻²⁰ moles/nm².

Next, the crosslinking agent is prepared by dissolving the dialdehyde in an aqueous solvent, preferably deionized water, then mixed with the previously prepared nanocompound in a concentration according to the used concentration of the linking agent, aiming for the molar concentration of the crosslinker to be equal to that of the linker. The resulting solution is left to stand for at least 60 minutes at a temperature of at least 4° C. At the end of this process, the crosslinking agent is anchored to the linker, which in turn is bound to the substrate.

Next, the INA protein is immobilized on the free end of the crosslinking agent, for which the INA protein is prepared by dissolving it in an aqueous solvent, preferably deionized water, at a concentration according to the concentration of the linking agent used, so that the molar concentration of the INA protein is equal to that of the linker, stirring the mixture for at least 2 minutes.

Then this INA protein solution is mixed with the nanocompound functionalized with the linker and crosslinking agent resulting from the previous step, stirring said mixture for a time of at least 1 minute. The resulting solution is left to stand for a minimum of 24 hours at a minimum temperature of 4° C. At the end of this process the INA protein is directly anchored to the crosslinking agent, which in turn is bound to the linker, which in turn is bound to the substrate forming the bio-nanocompound.

Optionally, the bio-nanocompound is washed in order to remove the excess material. For this purpose, an aqueous solvent, preferably deionized water, is added to the bio-nanocompound, the particle size of the bio-nanocompound is verified and according to this, the bio-nanocompound is filtered by mechanical or magnetic processes to recover the solid bio-nanocompound, then the filtered solid is mixed with more washing medium, stirring for 1 minute. The washing process is repeated as many cycles as necessary depending on the particle size.

### Coolant

The coolant of this disclosure is defined for purposes of this disclosure as a dispersion of the bio-nanocompound in an aqueous-based liquid medium. The bio-nanocompound when added to an aqueous-based solvent or water-like substance, functions as an additive that promotes freezing of the liquid. Additionally, it allows the improvement of the cooling capacity of such liquids, in terms of increasing the formation of ice nuclei at higher temperatures from the natural freezing point, making it easier to reach its solid state and allowing longer thawing time.

In a specific embodiment and not being a limitation, the water-based solvent is selected from any common coolant, specifically glycols or alcohols and more specifically water, ethanol, glycerol, or mixtures thereof.

In a specific embodiment, the concentration of the bio-nanocompound in the solvent is from 0.1 to 10% % w/w allowing the coolant to reach its solid state, promoting the formation of crystals and thus the rapid freezing of the total solvent.

Uses In a preferred mode the bio-nanocompound of this development is used to add coolants, whose uses are directed to applications in packaging, ventilation systems, wastewater treatment, agriculture, refrigeration equipment for sectors such as health, pharmaceutical, food, entertainment, sports, agriculture, among others, without being a limitation.

This disclosure is presented in detail through the following examples, which are provided for illustrative purposes only, and are not intended to limit its scope.

### EXAMPLES

### Example 1. Structure of the Bio-Nanocompound

### Substrates

Iron oxide substrate (Fe₃O₄):

One gram of magnetite (iron oxide) nanoparticles was synthesized with 0.545 g of ferric chloride and 1.394 g of ferrous chloride and placed in a beaker with 4.3 ml of Milli-Q water and a magnetic stirrer. This solution was subjected to heat treatment with a hot plate at 90° C. and 1500 rpm.

Subsequently, two solutions, the first of 3.3 g NaOH and 10 ml Milli-Q water and the second of 0.8 ml 25% TMAH by weight and 9.2 ml Milli-Q water, were drawn into two syringes and both were placed in the syringe pump, with an output rate of 0.2 ml/min. The 20-gauge probes connected to each syringe were placed in the beaker with the chloride solution and allowed to react for one hour.

The final solution was decanted with the aid of a magnet and the supernatant was removed. Ten ml of Milli-Q water were added and the solution was sonicated for 5 min. These steps were repeated 20 times until the magnetite nanoparticles were completely washed out.

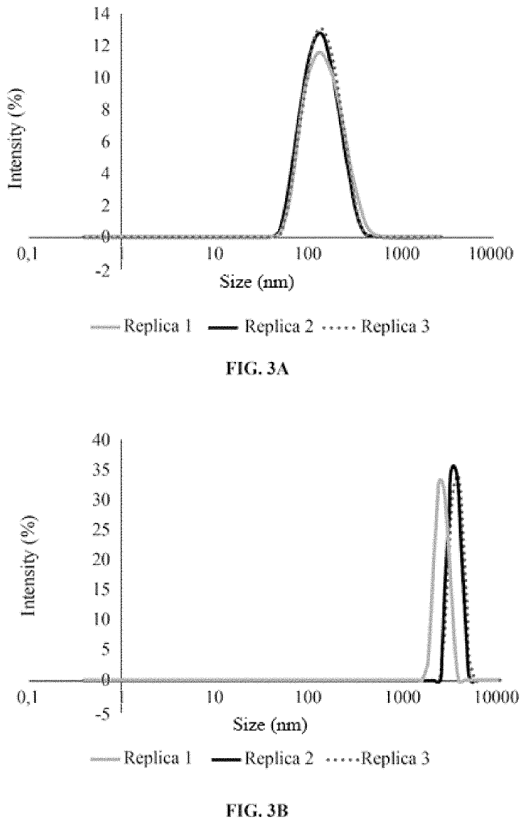

The synthesized magnetite nanoparticles were analyzed by Z Sizer Nano equipment. Three 1 ml replicas of the solution were placed in different cells. The average size of the nanoparticles was 132 nm, as evidenced in FIG. 3A.

Substrates of silicon dioxide (SiO₂) and aluminum (Al₂O₃):

The silicon and aluminum oxide nanoparticles were obtained from the market and then centrifuged at 4,000 rpm for 10 minutes. Subsequently, the supernatant was removed and 10 ml of Milli-Q water was added to subject the solutions to sonication for 5 minutes and vortexing for another five minutes. The procedure was repeated 20 times.

The silicon dioxide nanoparticles were measured with the Z Sizer Nano equipment by placing three 1 ml replicas of the solution. The average size obtained was 2,243 nm as shown in FIG. 3B.

The substrates obtained as indicated above, were functionalized with aminosilane 3-aminopropyl triethoxysilane (APTES), glutaraldehyde was added as a crosslinker and the INA protein was immobilized, as described below in Example 2. The structure of the bio-nanocompound obtained is as shown in FIG. 2. It is possible to observe the bonds obtained between the nanoparticles formed by the substrate, the linker (APTES), the crosslinking agent (glutaraldehyde) and the ice nucleation agent (the INA protein). It is evident that the INA protein is linked to the glutaraldehyde by an amino group that binds to the aldehyde residue of the crosslinking agent, the other aldehyde end of the crosslinking agent binds to the amino group of the linker, and the linker is directly attached to the substrate by a three-point chemical anchor.

### Example 2. General Method for Obtaining the Bio-Nanocompound

One hundred (100) mg of aluminum oxide, silicon dioxide and magnetite nanoparticles obtained, according to Example 1, were used as substrates to immobilize the ice nucleation active (INA) protein described in U.S. Pat. No. 6,151,902A.

### Aluminum Oxide

One hundred (100) mg of aluminum oxide nanoparticles, in an order of magnitude of 400 μm, were subjected to a self-assembly technique, in which APTES was attached as a linker with the aid of ultrasonic washing at 30° C. for 10 min. The product was allowed to stand for 10 min and then glutaraldehyde was added and bound to the linker molecules via the homobifunctional amino reaction.

### Magnetite and Silicon

One hundred (100) mg of magnetite and silicon dioxide nanoparticles, with an average size of 132 nm and 2,243 nm, respectively, were individually suspended in a solution of Milli-Q water and TMAH, to be subjected to ultrasonic washing for 20 min at 30° C. Then, these nanoparticles were treated in the same way as aluminum oxide nanoparticles, as mentioned above.

### Immobilization of Ice Nucleation Active Protein (INA)

A 10 mg/ml dilution of the INA protein (U.S. Pat. No. 6,151,902) was obtained using Milli-Q water which was added to the different functionalized surfaces after 60 minutes of incubation at 4° C. The solutions of the functionalized nanoparticles and the INA dilution were left for 24 hours at 4° C. to immobilize the proteins by a reaction analogous to the peptide condensation reaction between the amino groups of the peptides and the carbonyl groups of the glutaraldehyde.

### Example 3. Method for Obtaining an Iron Oxide Bio-Nanocompound

The preparation of the substrate was carried out by weighing 100 mg of iron oxide in an analytical balance and then mixing with ultrapure water (MQ) in an amount that would cover the substrate (5 ml). Following this, the particle size of the substrate was verified to be between 132 nm and 400 μm and a dispersant was prepared which is a 20% solution of TMAH in ultrapure water.

The dispersant was mixed with the substrate dissolved in water at a mg/μL ratio of 2:1 substrate:dispersant and the mixture was agitated in an ultrasonic bath for 20 minutes at 30° C. The addition of dispersant to the substrate was carried out when the substrate reached a particle size of nanometer order.

Subsequently, the linker was prepared by diluting APTES to a concentration of 1% in water, mixed with the previously prepared substrate solution or nano-sized substrate with TMAH, at a substrate linker concentration of 1:1 mg/μL; stirred in an ultrasonic bath for a time of 10 minutes at a temperature of 30° C. and allowed to stand for 10 minutes at a temperature of 15° C.

Next, the crosslinking agent was prepared by diluting glutaraldehyde to a concentration of 2% in water, mixed with the solution resulting from adding APTES to the substrate at a 1:1 mg/μL substrate:crosslinker ratio, stirred in an ultrasonic bath for 20 minutes and left to stand for one hour at a temperature of 4° C.

Next, the INA protein was prepared by weighing 10 mg of SNOMAX® and adding 1 ml of water until a ratio of 10:1 mg/mL INA protein/water was achieved and stirred for 2 minutes. Once the INA protein was prepared, it was added to the previous crosslinking agent-crosslinker-substrate mixture, stirred for 1 minute and left to stand for one day at 4° C.

Finally, the bio-nanocompound was washed by adding 5 mL of water and filtering with filter paper to recover the solid, which was mixed with more water at a ratio of 1:20 mg/mL and stirred for 1 minute, repeating the filtering and washing process 15 times until a substrate particle size of between 132 nm and 400 μm was achieved.

The general structural arrangement of the bio-nanocompound after being obtained through the method described herein is as shown in FIG. 2.

### Example 4. INA Protein Immobilization Efficiency

The immobilization efficiency of INA proteins on the substrate was measured as follows:

### Calibration Curve

The calibration curve was obtained to have a standard reference with increasing concentrations of bovine serum albumin (BSA) protein to localize the INA protein concentrations that were immobilized.

Six 100 μL dilutions with different concentrations of BSA (0, 1.25, 2.5, 5, 7.5 and 10 μg/ml) were placed in acrylic cells. Subsequently, 700 μL of Milli-Q water and 200 μL of Bradford reagent from the Bio-Rad protein assay were added to each of these cells. Bradford reagent is used to generate different shades of blue depending on the protein concentration due to a brown to blue color change arising from the binding of protein molecules to the Coomassie dye under acidic conditions.

After adding the Bradford reagent, the contents of each cell were mixed and then left to incubate at room temperature for 15 minutes. Subsequently, these cells were placed in the spectrophotometer at a frequency of 595 nm, in order to read the absorbance of the diluted protein. The BSA concentrations established allowed the corresponding calibration curve to be obtained.

### Sample Analysis

The supernatant from the three functionalization processes described in the foregoing examples was analyzed by Bradford assay using the calibration standard curve obtained above and three replicas for each consisting of: 700 μL of Milli-Q water, 100 μL of supernatant and 200 μL of Bradford reagent from the Bio-Rad protein assay. The replicas were allowed to stand for 8 minutes at room temperature and then analyzed with a spectrophotometer at 595 nm.

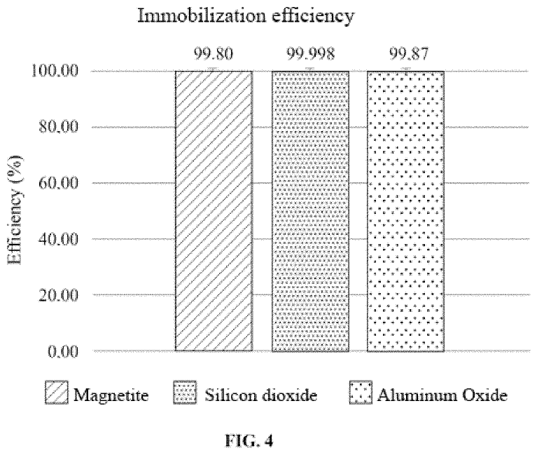

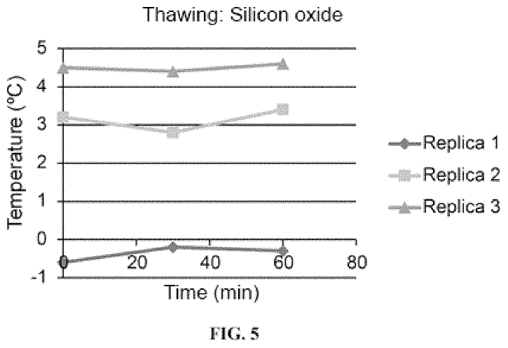

It was confirmed that the INA protein had been immobilized on the substrates by the immobilization efficiency, which was 99.99%, 99.87% and 99.80% for silicon dioxide, aluminum oxide and iron oxide substrates, respectively, which is related to the concentrations of INA protein in the supernatant of the immobilization solutions (FIG. 4). Therefore, it was determined that silicon dioxide nanoparticles have the highest amount of INA protein on their surface, followed by aluminum oxide and magnetite.

### Example 5. Freezing Activity of the Bio-Nanocompound

It was carried out in order to determine the temperature at which each of the samples showed ice nucleation activity.

### Sample Preparation

Samples A, B, C, D, E, F, G, H, I, J and K were prepared, as shown in Table 1, corresponding respectively to three dilutions of magnetite, three of silicon, three of free

INA protein and two of Milli-Q water control.

TABLE 1
Samples for freezing activity assays

| Sample | Substrate | Concentration |
|---|---|---|
| A | Magnetite | 2 mg/ml |
| B | Magnetite | 1 mg/ml |
| C | Magnetite | 0.5 mg/ml |
| D | Silicon | 2 mg/ml |
| E | Silicon | 1 mg/ml |
| F | Silicon | 0.5 mg/ml |
| G | Free INA protein | 0.2 mg/ml |
| H | Free INA protein | 0.1 mg/ml |
| I | Free INA protein | 0.05 mg/ml |
| J | Control - water | N/A |
| K | Control - water | N/A |

Five ml of three different concentrations of each of the bio-nanocompounds diluted in Milli-Q type I water were placed in 8 ml beakers and resuspended until a homogeneous dilution mixture was obtained. Afterwards, each beaker was closed with its respective stopper and treated in an ultrasonic bath. Each concentration sample was processed in triplicate for a total of 9 samples.

Moreover, a total of 5 mg of INA protein was diluted in 5 ml of Milli-Q water. Subsequently, the same concentrations of protein that the bio-nanocompound had immobilized were obtained with three additional dilutions. Five ml of three different concentrations of the previous dilution of free INA protein in Milli-Q water (G, H and I) were placed in 8 ml beakers and resuspended until a homogeneous dilution was obtained.

Afterwards, each beaker was closed with its respective stopper, but with their respective concentrations. Samples G and H had three replicas and concentration sample I had two replicas, for a total of 8 free INA protein samples.

Finally, two 5 ml samples of Milli-Q water (J and K) were placed in 8 ml beakers with their respective stoppers, serving as negative controls in the ice nucleation activity experiments.

Before starting each dilution, the bio-nanocompounds immobilized on magnetite and silicon dioxide were placed in an ultrasonic bath at 20° C. for 5 minutes and then vortexed for 30 seconds.

All dilutions (A, B, C, D, E, F, G, H, I, J, K) were reused to replica the experiments more than five times to evaluate the reusability of bio-nanocompounds as ice nucleators and free INA protein.

### Freezing Experiments

Five experiments were carried out to evaluate the freezing activity of each sample.

Experiment 1. The first experiment was carried out in a cooling chamber with temperatures ranging between 1.5° C. to 2.4° C.

One (1) ml of the serial dilutions of magnetite and silicon dioxide bio-nanocompounds (2 mg/ml, 1 mg/ml, 0.5 mg/ml, 0.25 mg/ml and 0.125 mg/ml) were placed in a cooling chamber with temperatures ranging from 1.5° C. to 2.4° C. for 4 hours to measure the freezing point of each concentration for the two types of bio-nanocompounds.

Three replicas per concentration of bio-nanocompounds were evaluated. The symbol (*) means ice nucleation activity and (-) means no nucleation activity or liquid state of the samples.

TABLE 2
Determination of ice nucleation activity for magnetite and silicon dioxide bio-nanocompounds.

| Magnetite | | | | Silicon Dioxide | | | |
|---|---|---|---|---|---|---|---|
| Concentration | | | | Concentration | | | |
| (mg/ml) | Replica 1 | Replica 2 | Replica 3 | (mg/ml) | Replica 1 | Replica 2 | Replica 3 |
| 2 | * | * | * | * | * | * | * |
| 1 | * | * | * | — | — | — | — |
| 0.5 | * | * | * | — | — | — | — |
| 0.25 | — | — | — | — | — | — | — |
| 0.125 | — | — | — | — | — | — | — |

(*) represents ice nucleation activity and (—) represents no nucleation activity or liquid state of the samples.

According to the foregoing results, the bio-nanocompound with the best ice nucleation behavior at 1.5° C.-2.4° C. is the magnetite bio-nanocompound, as 3 out of 5 concentrations froze. However, the silicon dioxide bio-nanocompound is better in terms of ice strength, because it was the only one that could be measured with this method.

Experiment 2. Five (5) ml of serial dilutions namely, 2 mg/ml, 1 mg/ml and 0.5 mg/ml and the free INA protein dilutions 0.2 mg/ml, 0.1 mg/ml and 0.05 mg/ml were placed in plastic containers with their respective caps and left in a cooling chamber at −7° C. until frozen. Then, the samples were subjected to a thermostatic bath at 26° C. to measure the state and temperature variation of each sample.

On the two occasions that this experiment was carried out, all samples froze due to the temperature to which they were subjected.

Experiment 3. All samples A, B, C, D, E, F, F, G, H, I, J and K, as described in the sample preparation were placed on a rack and placed in a cooling chamber at 4° C. for 12 hours to freeze them all and then evaluate the melting activity. At this temperature, the internal temperature of the freeze dryer was −1.1° C. and the water control did not freeze.

Experiment 4. All samples (A, B, C, D, E, F, F, G, H, I, J, K) were placed on a rack and placed in a cooling chamber at −5° C. for 12 hours to freeze and then evaluate their melting activity. At this temperature, the internal temperature of the freeze dryer was −2.8° C., so the water control did not freeze.

Experiment 5. All samples (A, B, C, D, E, F, F, G, H, I, J, K) were placed on a grid and placed in a cooling chamber at −6° C. for 12 hours to freeze them and then evaluate their melting activity. At this temperature, the internal temperature of the freeze dryer was −4.1° C. and the water control showed freezing activity.

### Example 6: Thawing Activity

Thawing experiments, corresponding to each of the freezing experiments in Example 5, were carried out.

Experiment 1. This experiment was carried out in order to compare the ice strength between the bio-nanocompounds, the free INA protein and the water control, by freezing all of them and then placing them at room temperature (23° C.), in order to measure the thawing time.

The thawing time of ice crystals promoted and generated by the bio-nanocompounds according to this disclosure, free INA protein and type I water control was measured by two methods. The first, consisted of freezing water samples at different concentrations of the bio-nanocompounds in a cooling chamber until ice crystals were present as described in Example 5, and then placing them in a thermostatic bath at 26° C., to record the temperature and state over time every 30 seconds until it reached the liquid state.

The second method involved placing water samples with different concentrations of bio-nanocompound in a freeze dryer for 12 hours, in order to assess whether any had frozen as described in Example 5, and then analyzing the frozen samples at room temperature (23° C.), measuring temperature and state over time every 30 seconds until they reached the liquid state.

The first method made it possible to measure the samples in seconds (FIG. 5), while the second method made it possible to measure for minutes in a more controlled environment.

After completion of the incubation period described in Example 5 from experiment 3, dilutions that showed ice nucleation activity, state 1 or 2 according to the conventions in Table 3 were placed at room temperature (23° C.), for one minute to test thawing activity and ice strength (FIG. 5). Magnetite samples were not included, since all samples were melted before any measurements were made.

TABLE 3
Table of State Conventions. The state of the samples was
determined according to the conventions in this table, to
complement the characterization of the bio-nanocompounds

| | | Mainly | Semi-solid / | Mainly | |
|---|---|---|---|---|---|
| State | Solid | solid | Semi-liquid | Liquid | Liquid |
| Convention | 1 | 2 | 3 | 4 | 5 |

The following results demonstrate the performance of bio-nanocompounds through the second method:

The iron oxide bio-nanocompound had the following thawing times, depending on the previous freezing concentration and activity:
at −1.1° C.: no freezing (2 mg/ml), 25 min (1 mg/ml) and 30 min (0.5 mg/ml),
at −2.8° C.: 33.33 min (2 mg/ml), 33.33 min (1 mg/ml) and 31.7 min (0.5 mg/ml,
finally, at −4.1° C.: 45 min (2 mg/ml), 50 min (1 mg/ml) and 48.3 min (0.5 mg/ml).

The silicon dioxide bio-nanocompound had the following thawing times, depending on concentration and previous freezing activity:
at −1.1° C.: 20 min (2 mg/ml), 30 min (1 mg/ml) and 30 min (0.5 mg/ml),
at −2.8° C.: 38.3 min (2 mg/ml), 38.3 min (1 mg/ml) and 36.7 min (0.5 mg/ml),
finally, at −4.1° C.: 43.3 min (2 mg/ml), 45 min (1 mg/ml) and 45 min (0.5 mg/ml).

Free INA protein had the following thawing times, depending on concentration and previous freezing activity:
at −1.1° C.: no freezing (0.2 mg/ml), 28.3 min (0.1 mg/ml) and 25 min (0.05 mg/ml),
at −2.8° C.: 33.3 min (0.2 mg/ml), 28.3 min (0.1 m /m1) and 35 min (0.05 mg/ml),
finally, at −4.1° C.: 45 min (0.2 mg/ml), 46.7 min (0.1 mg/ml) and 42.5 min (0.05 mg/ml).

A replica of the type I water control was frozen at −4.1° C. and lasted 30 minutes, until it reached the liquid state.

The foregoing demonstrates lower thawing times compared to those of the bio-nanocompound according to this disclosure, demonstrating an advantage of the bio-nanocompound over the controls reflected in lower energy usage for freezing and water maintenance.

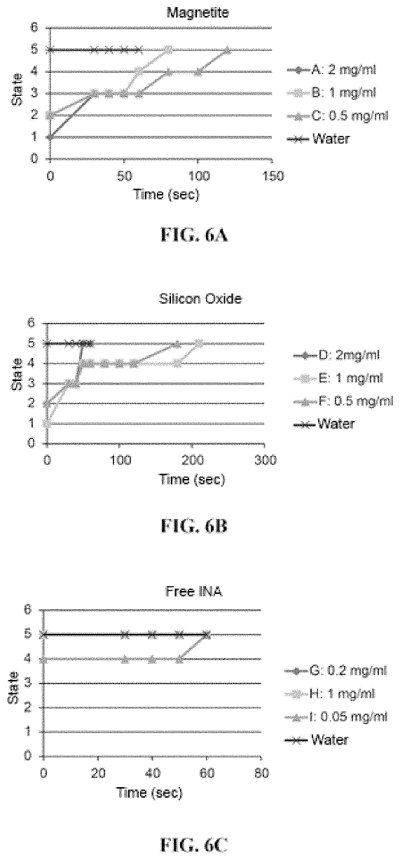

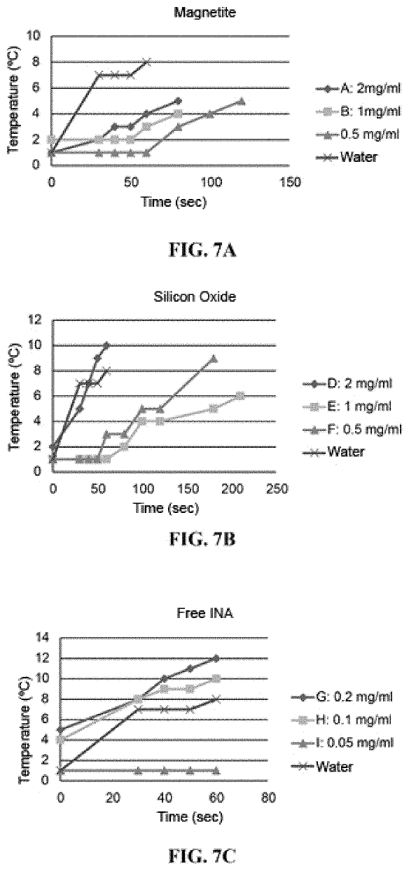

Experiment 2. Five (5) ml samples of serial dilutions frozen in a cooling chamber and subjected to a thermostatic bath at 26° C. were evaluated. Three replicas were included in each measurement. The results show the mean between the values of each replica for each sample. The water control had two replicas. The first time the experiment was conducted, state and temperature analyses were included and these are shown in FIGS. 6 and 7, respectively. Samples A to C correspond to magnetite (FIGS. 6A and 7A), D to F correspond to silicon dioxide (FIGS. 6B and 7B) and G to I correspond to free INA protein (FIGS. 6C and 7C).

The first time this experiment was carried out, no replicas were used. In this case, from FIGS. 6 and 7 it is possible to evidence that the silicon dioxide bio-nanocompound lasted 210 seconds, until it reached the liquid state with a difference of 90 seconds with the magnetite bio-nanocompound and 150 seconds with the free protein.

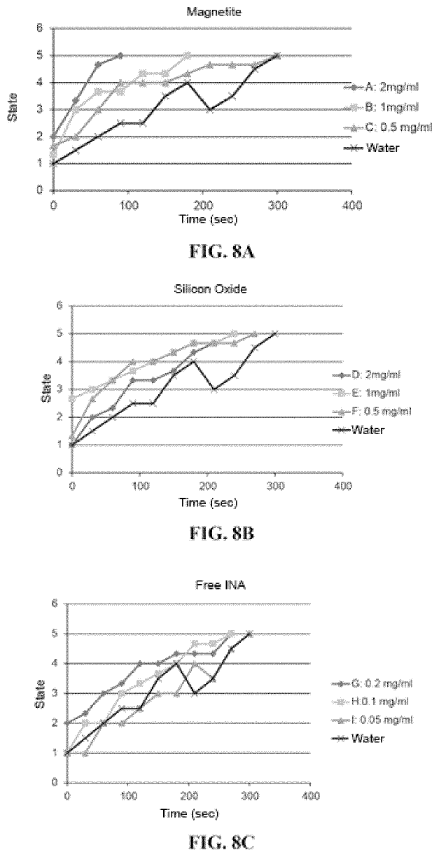

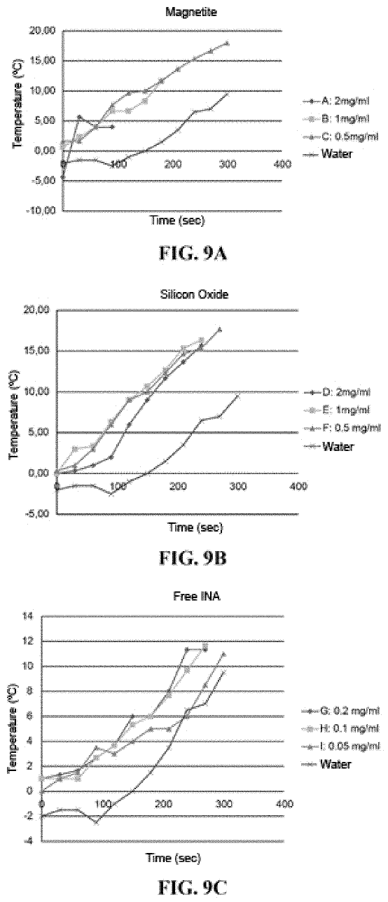

The results of the second time the experiment was carried out are shown in FIGS. 8 and 9, where three replicas were used for each treatment. In this case, the bio-nanocompound that presented the longest thawing time was magnetite at a concentration of 0.5 mg/ml, followed by silicon dioxide at a concentration of 0.5 mg/ml and free protein at a concentration of 0.05 mg/ml. Suggesting that both bio-nanocompounds exhibit good performance over time being at a lower concentration in solution.

Experiment 3. Samples were frozen and each concentration was evaluated independently by leaving the samples at room temperature (23° C.) after removal from the freeze dryer, testing only groups of 3 samples with the same concentration. Each beaker stopper had a hole with a diameter for the temperature sensor and the temperature of each sample was measured every 5 minutes with a thermocouple. At the same time, the solid/liquid state of the samples was determined using the preset convention from 1 to 5 according to Table 3.

The results of the experiment were reported in a timeline table providing an average state and deviation for each concentration sample. The results were then placed together in a graph to compare their respective ice strengths.

Sample A of the magnetite bio-nanocompound corresponding to a concentration of 2 mg/ml was not frozen at this temperature, like the type I water control and sample G of the INA protein, which corresponds to the concentration of 0.2 mg/ml. Therefore, there is no data representing these samples in FIG. 11. In this case the results of the magnetite sample are shown in FIG. 11A, those of silicon in FIG. 11B and those of free INA protein in FIG. 11C. The magnetite samples that perform best over time are those of 0.5 mg/ml, for the case of silicon dioxide the 0.1 mg/ml and 0.5 mg/ml concentration samples have similar performance resulting in the same time to reach the liquid state, and as for the free protein the 0.1 mg/ml samples take the longest time to thaw.

Since the 2 mg/ml magnetite sample was not frozen at −1.1° C., the bio-nanocompound with the best thawing activity behavior was silicon dioxide, since it has a thawing time of 30 minutes, for samples E and F, which is longer than that of sample B for magnetite (25 minutes), sample H for free INA (28 minutes) and sample I for free INA protein (25 minutes).

Moreover, the silicon dioxide bio-nanocompound shows better qualities than magnetite, free INA protein and water control, because it achieves the maximum thawing time even at the lowest concentration (0.5 mg/ml), which corresponds to the F sample.

Experiment 4. This experiment was carried out using the same parameters and conditions of the thawing activity of Experiment 3. The results of the magnetite sample are shown in FIG. 12A, those of the silicon dioxide in FIG. 12B and those of the free INA protein in FIG. 12C. By subjecting the samples to these conditions, magnetite presents a longer thawing time as it is present in a higher concentration (see samples A and B in the graph), silicon dioxide presents the same thawing time for the lower concentration samples (see samples E and F) and free INA protein presents a shorter thawing time compared to both bio-nanocompounds and longer thawing time at lower concentration (sample I).

At this temperature, the silicon dioxide bio-nanocompound presents a longer thawing time at higher concentration for samples D to F. Consequently, this bio-nanocompound has the best behavior over time, followed by the magnetite bio-nanocompound and the free INA protein.

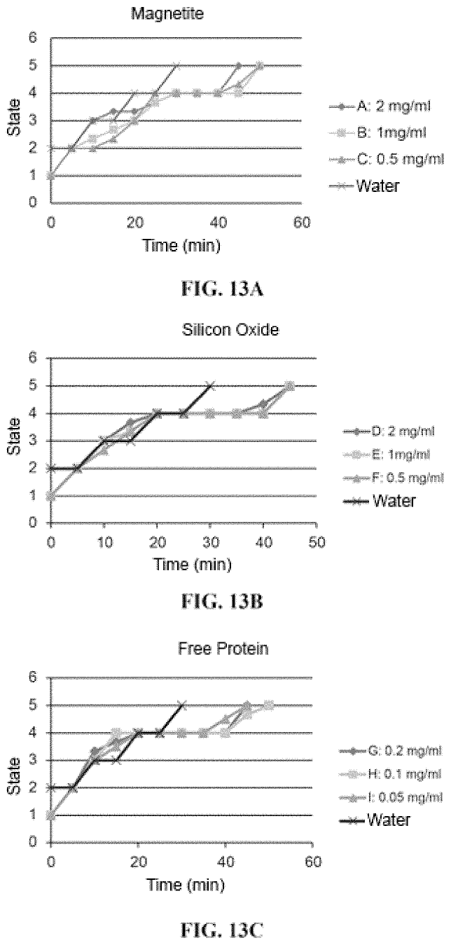

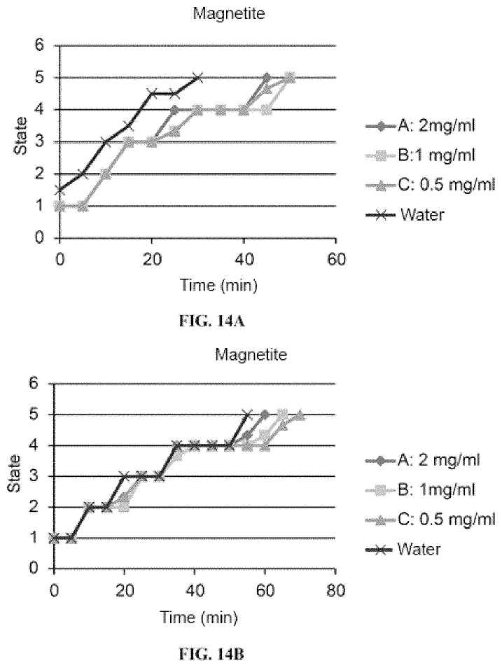

Experiment 5. This experiment was carried out using the same parameters and conditions for thawing activity as Experiment 3. The results for the magnetite sample are shown in FIG. 13A, those for silicon dioxide in FIG. 13B and those for free INA protein in FIG. 13C.

According to the results obtained under these freezing and thawing conditions, the magnetite bio-nanocompound has the longest thawing time and consequently the best ice resistance, followed by the silicon dioxide bio-nanocompound, which showed an increased thawing time at a lower concentration.

Free INA protein at a concentration of 0.1 mg/ml had a longer thawing time than silicon dioxide, by one minute difference, but had a shorter thawing time than both bio-nanocompounds at a concentration of 0.05 mg/ml.

Both bio-nanocompounds have a 15 to 20-minute thawing time advantage over the water control and the magnetite bio-nanocompound has a 5 to 8-minute thawing time advantage over the free INA protein.

### Example 7. Minimum Freezing Activity

Five (5) ml of all the different dilutions A, B, C, D, E, F, F, G, H, I, J and K were placed in plastic containers with their respective caps in a lyophilizer at temperatures of 4° C., 3° C., 2° C., 1° C., 0° C., -1° C., -2° C., -3° C., -4° C., -5° C., -6° C., -7° C. and -8° C., for 12 hours for each temperature and, then, the presence of the frozen state was evaluated in any of the samples. Three replicas of each sample and two replicas of type I water, defined as the negative control, were carried out.

There was no evidence of a frozen state in any of the bio-nanocompounds until the freeze dryer reached a temperature of −4° C. shown on the machine's electronic chart and an internal temperature of −1.1° C., measured with a mercury thermometer placed inside the freeze dryer. This indicates the actual temperature at which the frozen state was reached.

Specifically, the magnetite bio-nanocompound has the following minimum freezing temperatures, depending on the concentration: −2.8° C. (2 mg/ml), −2.23° C. (1 mg/ml), −1.67° C. (0.5 mg/ml).

Silicon dioxide bio-nanocompound has the following minimum freezing temperatures, depending on concentration: −1.67° C. (2 mg/ml), −1.1° C. (1 mg/ml), −1.1° C. (0.5 mg/ml).

On the other hand, free INA protein has the following minimum freezing temperatures, depending on the concentration: −4.1° C. (0.2 mg/ml), −1.1° C. (0.1 mg/ml) and −1.1° C. (0.05 mg/ml).

Finally, the type I system (water control) presented a minimum freezing temperature of −7.05° C.

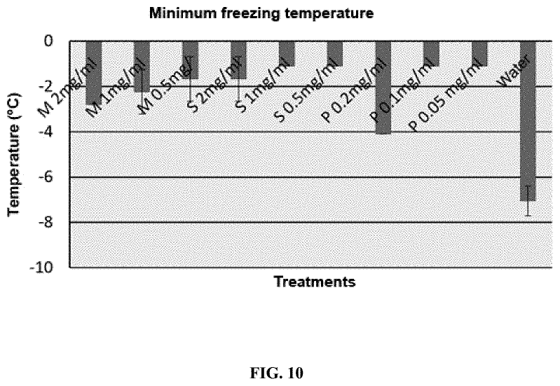

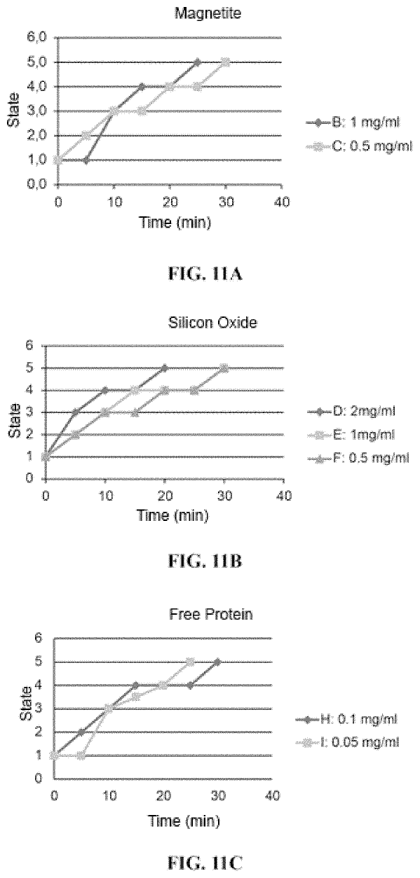

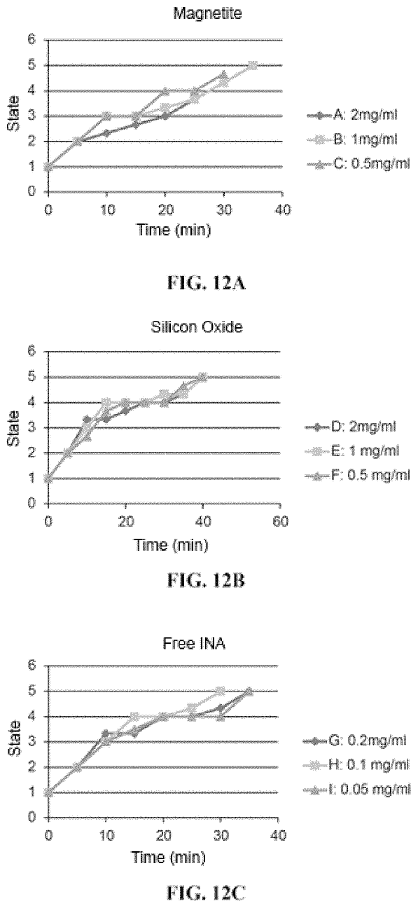

The results referenced above are illustrated in FIG. 10 and summarized in the following table:

TABLE 3
Minimum freezing temperature for bio-nanocompounds
of magnetite, silicon dioxide and free INA protein

| | | Concentration | Minimum freezing |
|---|---|---|---|
| Sample | Substrate | mg/ml | temperature (° C.) |
| A | Magnetite | 2 mg/ml | −2.8 |
| B | Magnetite | 1 mg/ml | −2.23 |
| C | Magnetite | 0.5 mg/ml | −1.67 |
| D | Silicon | 2 mg/ml | −1.67 |
| E | Silicon | 1 mg/ml | −1.1 |
| F | Silicon | 0.5 mg/ml | −1.1 |
| G | Free INA | 0.2 mg/ml | −4.1 |
| H | Free INA | 0.1 mg/ml | −1.1 |
| I | Free INA | 0.05 mg/ml | −1.1 |
| J | Control - water | N/A | −7.05 |

These results suggest that the bio-nanocompound with the highest freezing point is silicon dioxide, because the 3 replicas of 1 mg/ml and 0.5 mg/ml were frozen at −1.1° C. or −4° C. (freeze dryer temperature), while the magnetite bio-nanocompound was frozen at −1.67° C., at a concentration of 0.5 mg/ml. On the other hand, free INA protein alone was frozen at −4.1° C. and −1.1° C., at a concentration of 0.2 mg/ml and 0.1 mg/ml, respectively. The water control was frozen at −7.05° C.

The silicon dioxide bio-nanocompound presented a difference in its minimum freezing temperature of 5.38° C. and 2.43° C., with the water control and free INA protein, respectively.

The minimum freezing temperatures are consistent with the immobilization efficiency of the bio-nanocompound, as a higher amount of INA protein freezes water more efficiently.

### Example 8. Reusability of Bio-Nanocompounds as Ice Nucleators

This example was carried out to test the reusability of the bio-nanocompounds over time. Four cycles were run with the same bio-nanocompound samples, including free INA protein and type I water, with three replicas of each, with the exception of the free INA protein sample at a concentration of 0.05 mg/ml and type I water samples, which had only two replicas.

### Freezing Activity

All samples (A, B, C, D, E, F, G, H, I, J, K) were placed in the freeze dryer at −8° C. for 12 hours to freeze and then evaluate their thawing activity. Four cycles of this procedure were carried out. Under these conditions, the internal temperature of the freeze dryer was: −7.7° C., −8.1° C., −8.0° C. and −7.5° C. for cycles one through four. The water control showed freezing activity.

### Thawing Activity

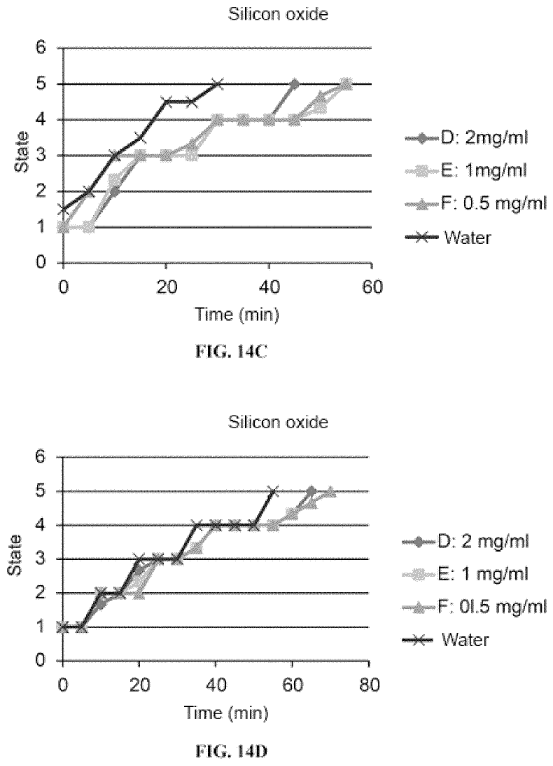

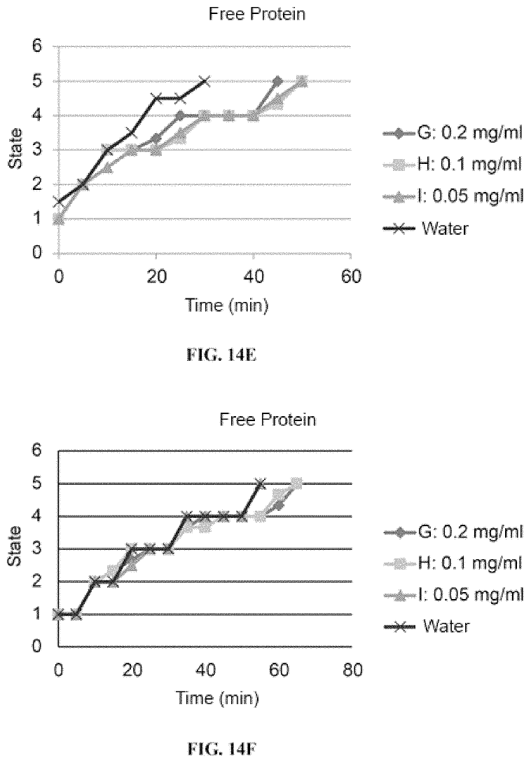

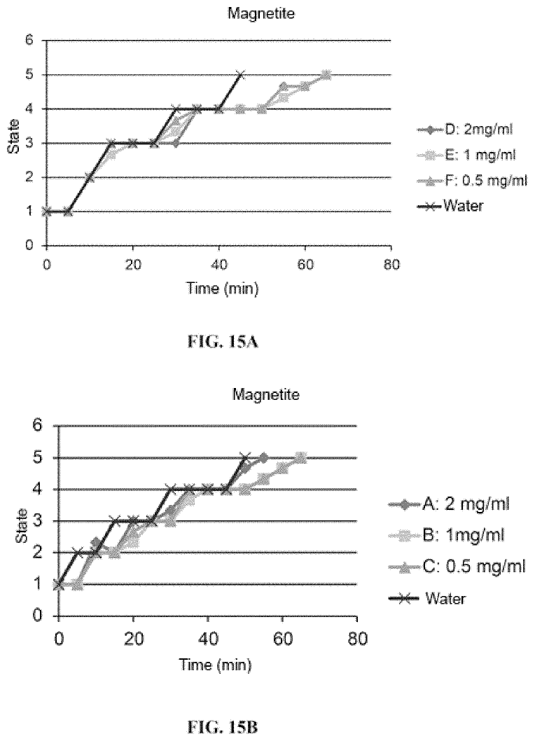

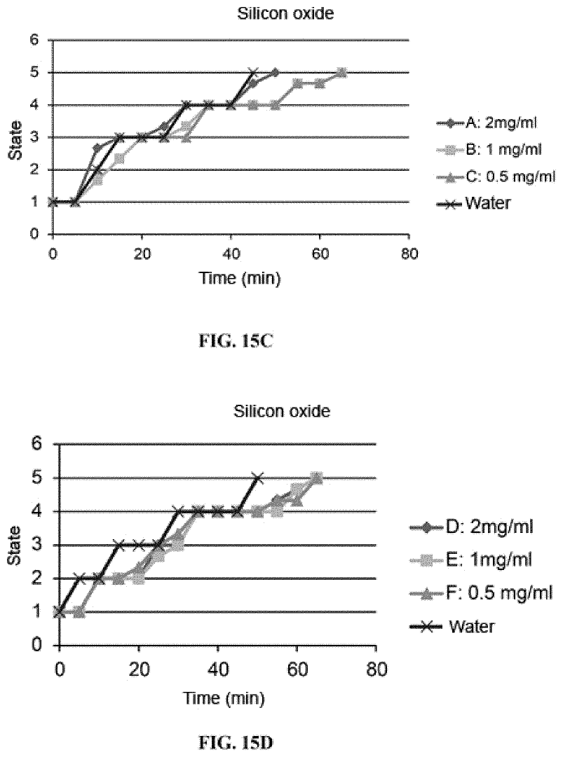

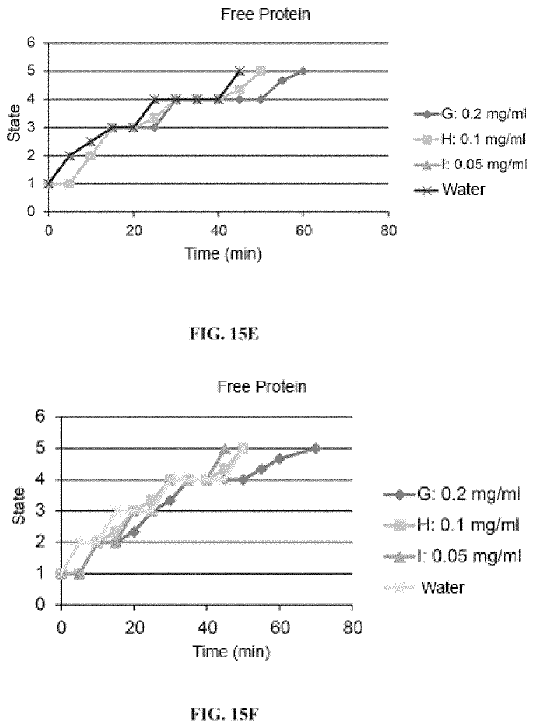

This experiment was carried out using the same parameters and conditions of the thawing activity described in Example 6. Therefore, the mean thawing time in minutes of the 3 replicas for each sample was plotted, with the water control being frozen under the aforementioned conditions (FIGS. 14 and 15). The results for the magnetite samples are shown in FIG. 14A, 14B, 15A and 15B, those for the silicon in FIGS. 14C, 14D, 15C and 15D, and those for the free INA protein in FIGS. 14E, 14F, 15E and 15F. In the four freezing cycles the bio-nanocompound and free protein samples present a longer thawing time than water. In the first cycle (FIGS. 14A, 14C and 14E) the concentration of the sample that presented the best performance was 1 mg/ml, which in the case of silicon dioxide took 55 minutes to reach the liquid state and 50 minutes in the case of magnetite and free protein. In the second cycle (FIGS. 14B, 14D and 14F) the concentration that presented the best behavior for the two bio-nanocompounds developed was 0.5 mg/ml, which took 70 minutes to reach the liquid state for both cases, while for the free protein the concentration that obtained the best time was 0.2 mg/ml with 65 minutes. In the third cycle (FIGS. 15A, 15C and 15E) the concentrations of 0.1 and 0.5 mg/ml are the ones that presented the longest thawing time for both bio-nanocompounds, with a time of 65 minutes for both magnetite and silicon dioxide, while the free INA protein presented a better time for the highest concentration (0.2 mg/ml) with a time of 60 minutes. Finally, in the fourth cycle (FIGS. 15B, 15D and 15F) the bio-nanocompounds presented the best thawing time for the lowest concentrations and the protein for the highest concentration, with times of 65 minutes for the bio-nanocompounds and 70 minutes for the free protein.

As is well known in the art, INA proteins degrade with temperature changes above 10° C. and do not guarantee a stable freezing temperature, thus generating problems of effectiveness and efficiency in the freezing process by having to use larger amounts of INA protein to achieve and maintain the desired freezing, as well as electrical energy to maintain a stable temperature. In this sense, according to the results obtained in the previous examples, it is evident that the specific structure of the bio-nanocompound developed through the self-assembly method, confers to the INA protein a strong bond to the substrate through a covalent chemical bond, which allows it to trap the protein through a bond that makes it very stable (greater thermal stability), avoiding its degradation and thus allowing a performance of this at different temperatures, which is characterized by presenting a permanent and lasting response to the freezing temperature.

On the other hand, the concentrations of the INA protein in the bio-nanocompound according to this disclosure are lower than the concentrations of the free protein, due to the immobilization process and its efficiency, corresponding to 99.99%, 99.87% and 99.80% for silicon dioxide, aluminum oxide and magnetite substrates, respectively. This means that the bio-nanocompound can generate ice nucleation with a smaller amount of protein than the free polypeptide alone.

Additionally, this structural feature and specifically the metallic substrate that allows to be collected by filtration or magnetic means, contributes to the recovery of the free INA protein, generating a cyclic operation implying that there is no loss of protein and therefore it can be given multiple uses, thus reducing the environmental impact, in addition to overcoming the drawbacks known in the prior art regarding the difficulty in reusing INA proteins for freezing processes and likewise, the high costs associated with the higher requirement in the amounts of INA protein. Moreover, the concentration of the protein assembled on the substrate can be increased locally, avoiding that the INA protein remains free and a stable freezing temperature cannot be guaranteed, thus providing greater refrigeration capacity by not remaining free; prolonging the cold chain, i.e., increasing the time in which the refrigerant liquid is frozen.

In addition, the bio-nanocompound allows for reduced energy consumption in that less electrical energy is required to reach the freezing point of the substance, since the bio-nanocompound allows nucleating crystals of the liquid at temperatures higher than the freezing temperature, thus requiring less cooling, and lasts longer frozen. Specifically, this disclosure provides a more efficient, resistant and durable ice nucleation agent at temperatures down to 0° C. leading to a decrease in energy consumption during ice formation. The bio-nanocompound of the disclosure retains high activity during the first three uses, after the third use, the activity decreases. However, the use of the bio-nanocompound provides a more stable cold chain because it provides a higher melting point and can be reused for a number of times, optimizing INA protein consumption.

In this sense, the cooling product has the advantage of reducing the loss of products due to breakage of the cold chain, reducing energy consumption by allowing freezing at higher temperatures (e.g., water freezes at 1.5-2.4° C. (34.7-36.3° F.), the bio-nanocompound can be reused several times allowing nucleating process to be repeated under conditions of low energy consumption, (e.g., the bio-nanocompound freezes up to four times), making it environmentally friendly, improves the crystallization process allowing the water to freeze at a faster rate and last longer due to its stronger bonds.

Finally, it is important to highlight that the bio-nanocompound provides a sustainable technology, since it promotes thermal stability, reducing energy consumption by approximately 80%, since, as has been emphasized, less energy is needed to reach the freezing point and the ice formed has longer thawing times.

1. A bio-nanocompound comprising:
a substrate of metal oxide particles in the nanometer to submillimeter size range;
a linker of an amino organosilane;
a dialdehyde crosslinking agent or crosslinker; and
an ice nucleation agent or INA protein,
wherein the linker is directly attached to the substrate, the dialdehyde crosslinking agent is directly attached to the linker and the ice nucleation agent is directly attached to the crosslinker.

2. The bio-nanocompound according to claim 1, wherein the metal oxide particulate substrate is selected iron, aluminum or silicon oxides.

3. The bio-nanocompound according to claim 1, wherein the linker is selected from (3-aminopropyl)triethoxysilane (APTES) or (aminopropyl)trimethoxysilane (APTMS).

4. The bio-nanocompound according to claim 1, wherein the crosslinking agent is selected from glutaraldehyde, succinaldehyde or glyoxal.

5. A self-assembly method for making the bio-nanocompound according to claim 1, comprising:
a) mixing the metal oxide substrate with a particle size in the nanometer to submillimeter range with a solvent medium;
b) immobilizing an amino organosilane linker on the substrate of step a);
c) immobilizing a dialdehyde crosslinker on the linker immobilized in step b); and
d) immobilizing by covalent bonding an INA protein or ice nucleation agent on the crosslinker immobilized in step c), thereby obtaining the bio-nanocompound according to claim 1.

6. A coolant comprising the bio-nanocompound according to claim 1 and an aqueous based solvent.

7. The coolant according to claim 6 wherein the water-based solvent is selected from the group consisting of water, glycerol and ethanol.

8. A method for nucleating aqueous-based compounds comprising applying the bio-nanocompound according to claim 1 to aqueous-based compounds.

9. A cooling additive comprising the bio-nanocompound according to claim 1.

10. A refrigeration equipment comprising the coolant according to claim 6.
